(19) United States
(12) Patent Application Publication
(10) Pub. No.: US 2011/0158927 A1
(43) Pub. Date: Jun. 30, 2011

(54) COSMETIC COMPOSITION COMPRISING AT LEAST ONE ORGANOSILICON COMPOUND, AT LEAST ONE ANIONIC SURFACTANT AND AT LEAST ONE CATIONIC POLYMER

(76) Inventors: Valérie VIRAVAU, Paris (FR); Carine Aires, Paris (FR); Damien Drillon, Paris (FR)

(21) Appl. No.: 12/977,116

(22) Filed: Dec. 23, 2010

Related U.S. Application Data
(60) Provisional application No. 61/298,651, filed on Jan. 27, 2010.

(30) Foreign Application Priority Data
Dec. 23, 2009 (FR) 0959498

(51) Int. Cl. A61K 8/72; A61K 8/73; A61K 8/81; A61K 8/89; A61Q 5/12; A61Q 5/02

(52) U.S. Cl. 424/70.12; 424/70.11; 424/70.13; 424/70.15

(57) ABSTRACT

The present disclosure relates to a cosmetic composition for washing and conditioning keratin fibers, including human keratin fibers such as the hair, comprising, in a cosmetically acceptable medium: (i) at least one organosilicon compound chosen from silanes comprising one silicon atom and siloxanes comprising two or three silicon atoms, the organosilicon compounds also comprising at least one basic chemical functional group and at least one group chosen from hydroxyl and hydrolyzable groups per molecule; (ii) at least one anionic surfactant; and (iii) at least one cationic polymer.

This application claims benefit of U.S. Provisional Application No. 61/298,651, filed Jan. 27, 2010. This application also claims benefit of priority under 35 U.S.C. §119 to French Patent Application No. 0959498, filed Dec. 23, 2009.

The present disclosure relates to a cosmetic composition for washing and conditioning keratin fibers, for example human keratin fibers, such as the hair, comprising, in a cosmetically acceptable medium, at least one organosilicon compound, at least one anionic surfactant and at least one cationic polymer. Also disclosed herein is a cosmetic process for treating keratin fibers, and also to a use employing the said cosmetic composition.

It is common practice to use detergent compositions, such as shampoos, based essentially on standard surfactants including anionic, nonionic and/or amphoteric type, such as anionic type-surfactants, for cleansing and/or washing keratin materials such as the hair. These compositions are applied to wet hair and the lather generated by massaging or frictioning with the hands makes it possible, after rinsing with water, to remove the diverse types of soiling initially present on the hair or the skin.

Although these base compositions may have good washing power, their intrinsic cosmetic properties are quite poor, for example due to the fact that the relatively aggressive nature of such a cleansing treatment may in the long term give rise to pronounced damage on hair fibers associated for instance with the gradual removal of the fats or proteins contained in or at their surface.

Thus, to improve the cosmetic properties of the above detergent compositions, for example detergent compositions that are required to be applied to sensitized hair (i.e. hair that is generally damaged or embrittled by the action of external atmospheric agents such as light and bad weather, and/or mechanical or chemical treatments such as blow-drying, combing, dyeing, bleaching, permanent-waving and/or relaxing), it is now common practice to introduce into these compositions additional cosmetic agents known as conditioning agents, which are intended mainly to repair or limit the harmful or undesirable effects caused by the various treatments or attacking factors to which the hair fibers are repeatedly subjected. These conditioning agents may also improve the cosmetic behavior of natural hair.

Known conditioning agents in the art, for example, cosmetically active organic compounds such as cationic polymers and silicones may be used in detergent cosmetic compositions such as shampoos in order to give the hair satisfactory cosmetic properties, for example in terms of sheen, softness, suppleness, lightness, a natural feel and improved disentangling.

However, the use of these compounds in cosmetic washing and hair-conditioning compositions may not give the hair satisfactory and long-lasting styling properties. Specifically, compositions comprising these compounds may result in insufficient styling effects, such as hair hold, body and/or manageability effects, and they may also have a tendency to fade out after washing the hair with a standard shampoo.

Thus, there is a need for washing compositions that are not only capable of appropriately conditioning the hair, but also capable of affording satisfactory and long-lasting styling effects.

Thus, compositions for washing and conditioning the hair comprising at least one organosilicon compound, such as 3-aminopropyltriethoxysilane, may make it possible to condition the hair, for example by giving it a satisfactory soft feel, while at the same time imparting pronounced and long-lasting styling effects. Furthermore, these compositions may also facilitate the shaping of fine hair and give beneficial styling effects to wavy or curly hair, for instance by improving the fashioning and control of the curls.

However, washing compositions comprising such organosilicon compounds may have the drawback of changing substantially over time under normal storage conditions as a function of the temperature, for example the compositions' viscosity and visual appearance may change. In other words, these compositions may not be stable, which may be reflected by a cloudy visual aspect and also by an unsatisfactory texture on storage.

Specifically, it has been found that organosilicon compounds, such as 3-aminopropyltriethoxysilane, are essentially chemically incompatible with surfactants, for example anionic surfactants, which may be present in washing compositions, leading to the stability problems encountered.

Moreover, it has been observed that the introduction of certain organosilicon compounds, for example amino derivatives such as 3-aminopropyltriethoxysilane, into washing compositions that generally have a pH ranging from 4 to 7 may also give rise to stability problems due to the alkaline nature of these compounds.

Thus, there is a need to develop cosmetic compositions for cleansing and conditioning keratin fibers containing organosilicon compounds that avoid at least one of the drawbacks described above, i.e. may be stable over time and may allow hair to be conditioned satisfactorily while at the same time affording long-lasting, powerful styling effects, for example in terms of volume, body and texturizing of the hair.

The present disclosure therefore relates to compositions for washing and conditioning keratin fibers, which have the desired properties, comprising at least one organosilicon compound as defined herein, at least one anionic surfactant and at least one cationic polymer.

Specifically, it has been found that the use of at least one cationic polymer in cosmetic compositions comprising at least one organosilicon compound and at least one anionic surfactant, may makes it possible to render the compositions stable during storage both at room temperature (20-25° C.) and at 45° C., for example with respect to the compositions' visual appearance and viscosity.

As used herein, “stable” is understood to mean that the visual appearance and viscosity of these compositions do not change substantially over time under standard storage test conditions, for example at room temperature (20° C.-25° C.), such as at 45° C. for two months following their manufacture.

Furthermore, the compositions in accordance with the present disclosure may lead to satisfactory treatment of the hair, thus giving it a satisfactory soft feel, improved disentangling, softness and suppleness. The disclosed compositions may also afford at least one powerful styling effect, such as providing volume, body and/or manageability for a lasting duration, and may also facilitate the shaping of the hair, for example the shaping of fine hair, and give improved styling effects to curly hair, for instance in terms of the fashioning and control of the curls for a lasting duration.

Thus, one aspect of the present disclosure relates to a cosmetic composition for washing and conditioning keratin fibers, for instance human keratin fibers such as the hair, comprising, in a cosmetically acceptable medium:

(i) at least one organosilicon compound chosen from silanes comprising one silicon atom and siloxanes comprising two or three silicon atoms, and wherein the at least one organosilicon compound also comprises at least one basic chemical functional group and at least one group chosen from hydroxyl and hydrolysable groups per molecule;

(ii) at least one anionic surfactant, and

(iii) at least one cationic polymer.

Also disclosed herein is a cosmetic process for treating keratin fibers, such as for washing and conditioning the hair, comprising applying to the keratin fibers a composition according to the present disclosure.

Another aspect of the present disclosure is a cosmetic process for treating keratin fibers, such as the hair, comprising applying an effective amount of a composition as described herein to the fibers, followed by rinsing it out after an optional leave-on time

Yet another aspect of the present disclosure relates to the use of the compositions disclosed herein as a shampoo for washing and conditioning the hair.

The at least one organosilicon compound (i) used in the compositions according to the present disclosure is chosen from organosilanes comprising one silicon atom and organosiloxanes comprising two or three silicon atoms, such as two silicon atoms. The at least one organosilicon compound (i) must also comprise at least one basic chemical functional group, for instance only one basic chemical functional group. The basic chemical functional group may correspond to any function that gives the silicon compound a basic nature, and in at least one embodiment is chosen from amine functional groups such as a primary, secondary and tertiary amine functional groups. The silicon compounds according to the present disclosure may optionally comprise other functional groups, for instance acid functional groups and halogen functional groups.

The at least one organosilicon compound (i) in the compositions disclosed herein further comprise at least one group chosen from hydrolysable and hydroxyl groups per molecule. In at least one embodiment of the present disclosure, the hydrolysable groups are chosen from alkoxy, aryloxy and halogen groups. The at least one organosilicon compound (i) may also optionally comprise other chemical functional groups such as acid functional groups.

According to at least one embodiment of the present disclosure, the at least one organosilane used in the compositions disclosed herein are chosen from compounds of formula (I):

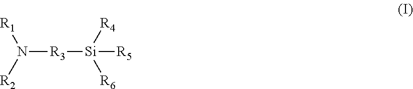

wherein:

R₄ is chosen from halogen atoms, OR′, and R′₁;

R₅ is chosen from halogen atoms, OR″, and R′₂;

R₆ is chosen from halogen atoms, OR′″, and R′₃; and

R₁, R₂, R₃, R′, R″, R′″, R′₁, R′₂ and R′₃ are each independently chosen from saturated and unsaturated, linear and branched hydrocarbon-based groups optionally bearing additional chemical groups; or in the alternative, R₁, R₂, R′, R″ and R′″ may also be a hydrogen atom, and wherein at least two of the groups R₄, R₅ and R₆ are chosen from OR′, OR″ and OR′″, and at least two of the groups R′, R″ and R′″ are other than hydrogen.

In another embodiment of the present disclosure, R₁, R₂, R′, R′₁, R′₂, R′₃, R″ and R′″ are each independently chosen from C₁-C₁₂ alkyl, C₆-C₁₄ aryl, (C₁-C₈)alkyl(C₆-C₁₄)aryl and (C₆-C₁₄)aryl(C₁-C₈)alkyl radicals.

According to at least one embodiment, the at least one organosiloxane used in the compositions disclosed herein are chosen from compounds of formula (II):

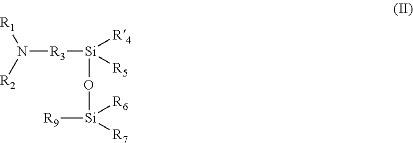

wherein:

R₁, R₂, R₃, R₅ and R₆ are as defined previously;

R′₄ is chosen from halogen atoms and OR₁₁;

R₇ is chosen from halogen atoms, OR₁₀, and R″₁;

R₉ is chosen from halogen atoms, OR₈, R″₂ and R₃NR₁R₂; and

R″₁, R″₂, R₈, R₁₀ and R₁₁ are each independently chosen from saturated and unsaturated, linear and branched hydrocarbon-based groups, optionally bearing additional chemical groups, or in the alternative, R₁₁, R₁₀ and R₈ may also be a hydrogen atom; and wherein at least one of the groups R₆, R₇ and R₉ is chosen from halogen atoms, OR′″, OR₁₀ and OR₈.

In at least one embodiment, the groups R″₁, R″₂, R₈ or R₁₀ and R₁₁ are chosen from C₁-C₁₂ alkyl, C₆-C₁₄ aryl, (C₁-C₈)alkyl(C₆-C₁₄)aryl and (C₆-C₁₄)aryl(C₁-C₈)alkyl radicals.

In at least one embodiment of the present disclosure, the halogen atom is a chlorine atom.

According to at least one embodiment, the at least one organosilicon compound used in the compositions disclosed herein are organosilanes chosen from compounds of formula (III):

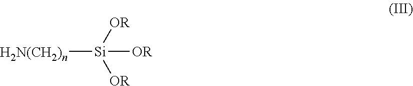

wherein each instance of R is independently chosen from C₁-C₆ alkyl radicals, such as C₁-C₂ alkyl radicals, and n is an integer ranging from 1 to 6, for example ranging from 2 to 4.

Other suitable silanes and siloxanes include those that are water-soluble, for instance, silanes and siloxanes that are soluble to a concentration of 2%, such as soluble to a concentration of 5%, for example, soluble to a concentration of 10%, by weight in water at a temperature of 25° C.±5° C. and at atmospheric pressure. As used herein, the term “soluble” is understood to mean the formation of a single macroscopic phase.

According to at least one embodiment of the present disclosure, the at least one organosilicon compound (i) is 3-aminopropyltriethoxysilane.

The at least one organosilicon compound (i) may be present in the compositions disclosed herein in an amount ranging from 0.01% to 10% by weight, for example, ranging from 0.1% to 5% by weight, such as ranging from 0.2% to 2% by weight, relative to the total weight of the composition.

The at least one anionic surfactant (ii) used in the compositions disclosed herein include, but are not limited to salts, for example alkali metal salts such as sodium salts, ammonium salts, amine salts, amino alcohol salts; and alkaline-earth metal salts, for example magnesium salts; alkyl sulfates; alkyl ether sulfates; alkylamido ether sulfates; alkylarylpolyether sulfates; monoglyceride sulfates; alkylsulfonates; alkylamidesulfonates; alkylarylsulfonates; α-olefin sulfonates; paraffin sulfonates; alkylsulfosuccinates; alkyl ether sulfosuccinates; alkylamidesulfosuccinates; alkyl sulfoacetates; acylsarcosinates and acylglutamates; wherein the alkyl and acyl groups of all these compounds comprise from 6 to 24 carbon atoms and the aryl group is chosen from phenyl and benzyl groups.

It is also possible to use monoesters of C₆-C₂₄ alkyl and of polyglycoside dicarboxylic acids, such as alkyl glucoside citrates, alkyl polyglycoside tartrates and alkyl polyglycoside sulfosuccinates, alkyl sulfosuccinamates, acylisethionates and N-acyltaurates, wherein the alkyl or acyl group of all these compounds comprise 12 to 20 carbon atoms.

Other suitable examples of anionic surfactants include acyl lactylates, wherein the acyl group comprises 8 to 20 carbon atoms.

In addition, non-limiting mention may also be made of alkyl-D-galactosideuronic acids and salts thereof, polyoxyalkylenated (C₆-C₂₄)alkyl ether carboxylic acids, polyoxyalkylenated (C₆-C₂₄)alkyl(C₆-C₂₄)aryl ether carboxylic acids, and polyoxyalkylenated (C₆-C₂₄)alkylamido ether carboxylic acids and salts thereof, for example those comprising 2 to 50 ethylene oxide units, such as 2 to 10 ethylene oxide units, for instance 2 to 5 ethylene oxide units, and mixtures thereof.

Alkyl sulfates, alkyl ether sulfates and alkyl ether carboxylic acids, and mixtures thereof, for example in the form of alkali metal or alkaline-earth metal, ammonium, amine or amino alcohol salts are used in at least one embodiment of the present disclosure.

According to at least one embodiment of the present disclosure, the at least one anionic surfactant (ii) used in the cosmetic compositions disclosed herein is chosen from alkyl ether sulfates, such as C₁₂-C₁₄ alkyl ether sulfates, and alkyl ether sulfates comprising from 2 to 3 mol of ethylene oxide.

The at least one anionic surfactant (ii) in the cosmetic compositions disclosed herein are present in a total amount ranging from 1% to 25% by weight, such as in an amount ranging from 3% to 20% by weight, for example in an amount ranging from 5% to 15% by weight, relative to the total weight of the cosmetic composition.

In at least one embodiment of the present disclosure, the at least one anionic surfactant (ii) is present in a total amount greater than at least 4% by weight relative to the total weight of the cosmetic composition.

As indicated previously, the cosmetic composition according to the present disclosure also comprises at least one cationic polymer (iii).

As used herein, the term “cationic polymer” is understood to mean any polymer containing cationic groups and/or groups that may be ionized into cationic groups.

The at least one cationic polymer (iii) present in the compositions according to the present disclosure may be chosen from any of those already known in the hair art, for example, the cationic polymers disclosed in European Patent Application No. 0 337 354 and in French Patent Nos. FR-2 270 846, 2 383 660, 2 598 611, 2 470 596 and 2 519 863.

According to the present disclosure, the at least one cationic polymer (iii) has a number-average molecular mass ranging from 500 to 5×10⁶ such as ranging from 10³ to 3×10⁶.

Among the cationic polymers that may be mentioned include but are not limited to polymers of the polyamine, polyamino amide and polyquaternary ammonium type, for example the cationic polymers disclosed in French Patent Nos. 2 505 348 and 2 542 997.

The at least one cationic polymer (iii) used in the cosmetic composition according to the present disclosure may also be chosen in a non-limiting manner from:

(1) Homopolymers or copolymers derived from acrylic or methacrylic esters or amides and comprising at least one of the units of formulae (IV), (V), (VI) or (VII) below:

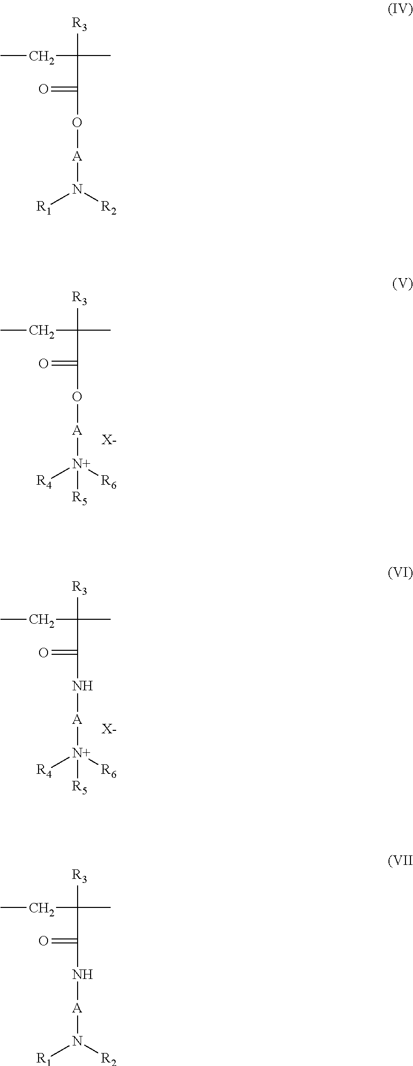

wherein:

each instance of R₃, is independently chosen from a hydrogen atom and a CH₃ radical;

each instance of A, is independently chosen from linear and branched C₁-C₆ alkyl groups, such as C₂-C₃ alkyl groups, and hydroxylalkyl group comprising 1 to 4 carbon atoms;

R₄, R₅ and R₆, are each independently chosen from C₁-C₁₈ alkyl groups and benzyl radicals, and in at least one embodiment, C₁-C₆ alkyl groups;

R₁ and R₂, are each independently chosen from a hydrogen atom and C₁-C₆ alkyl groups, such as methyl and ethyl;

X is an anion derived from an inorganic or organic acid, such as a methosulfate anion or a halide such as chloride or bromide.

Non-limiting mention may be made of the ethyltrimethylammonium methacrylate chloride homopolymer.

The polymers of family (1) can also contain at least one unit derived from comonomers which may be chosen from the family of acrylamides, methacrylamides, diacetone acrylamides, acrylamides and methacrylamides substituted on the nitrogen with lower (C₁-C₄) alkyls, acrylic and methacrylic acids and esters thereof, vinyllactams such as vinylpyrrolidone and vinylcaprolactam, and vinyl esters.

Thus, among the polymers of family (1), non-limiting mention may be made of:

copolymers of acrylamide and of dimethylaminoethyl methacrylate quaternized with dimethyl sulfate or with a dimethyl halide, such as the product sold under the name HERCOFLOC by the company Hercules,

the copolymers of acrylamide and of methacryloyloxyethyltrimethylammonium chloride described, for example, in European Patent Application No. 0 80 976, and sold under the name BINA QUAT P 100 by the company Ciba Geigy,

the copolymer of acrylamide and of methacryloyloxyethyltrimethylammonium methosulfate sold under the name RETEN by the company Hercules,

quaternized and non-quaternized vinylpyrrolidone/dialkylaminoalkyl acrylate and methacrylate copolymers, such as the products sold under the name GAFQUAT by the company ISP, such as, for example, GAFQUAT 734 or GAFQUAT 755, or alternatively the products known as Copolymer 845, 958 and 937, which are disclosed in French Patent Nos. 2 077 143 and 2 393 573,

dimethylaminoethyl methacrylate/vinylcaprolactam/vinylpyrrolidone terpolymers, such as the product sold under the name GAFFIX VC 713 by the company ISP,

vinylpyrrolidone/methacrylamidopropyldimethylamine copolymers sold for instance under the name STYLEZE CC 10 by ISP, and

quaternized vinylpyrrolidone/dimethylaminopropylmethacrylamide copolymers such as the product sold under the name GAFQUAT HS 100 by the company ISP.

crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, for example methylenebisacrylamide. Non-limiting examples include crosslinked acrylamide/methacryloyloxyethyltrimethylammonium chloride copolymer (20/80 by weight) in the form of a dispersion containing 50% by weight of the said copolymer in mineral oil, sold under the name SALCARE® SC 92, and crosslinked methacryloyloxyethyltrimethylammonium chloride homopolymer containing about 50% by weight of the homopolymer in mineral oil or in a liquid ester, which are, for instance sold under the names SALCARE® SC 95 and SALCARE® SC 96 by the company Ciba.

(2) The cellulose ether derivatives containing quaternary ammonium groups, disclosed in French Patent No. 1 492 597, and those sold, for example, under the names UCARE POLYMER “JR” (JR 400, JR 125 and JR 30M) or “LR” (LR 400 or LR 30M) by the company Amerchol. These polymers are also defined in the CTFA dictionary as quaternary ammoniums of hydroxyethylcellulose that have reacted with an epoxide substituted with a trimethylammonium group.

(3) Cellulose copolymers or cellulose derivatives grafted with a water-soluble monomer of quaternary ammonium, for example, those disclosed in U.S. Pat. No. 4,131,576, such as hydroxyalkylcelluloses, for instance hydroxymethyl-, hydroxyethyl- and hydroxypropyl-celluloses grafted, for instance, with a methacryloylethyl-trimethylammonium, methacrylamidopropyltrimethylammonium or dimethyldiallylammonium salt.

Other suitable non-limiting examples include the commercial products sold under the names CELQUAT L 200 and CELQUAT H 100 by the company National Starch.

(4) The cationic guar gums described for instance in U.S. Pat. Nos. 3,589,578 and 4,031,307, such as guar gums containing cationic trialkylammonium groups. Guar gums modified with a salt (e.g. chloride) of 2,3-epoxypropyltrimethylammonium may also be used in the compositions disclosed herein.

Suitable examples of cationic guar gums include those sold under the trade names JAGUAR C13 S, JAGUAR C 15, JAGUAR C 17 or JAGUAR C162 by the company Rhodia.

(5) Polymers formed from piperazinyl units and divalent alkylene or hydroxyalkylene radicals containing straight or branched chains, optionally interrupted with oxygen, sulfur or nitrogen atoms or with aromatic or heterocyclic rings, and also the oxidation and/or quaternization products of these polymers. Such polymers are described, for instance, in French Patent Nos. 2 162 025 and 2 280 361.

(6) Water-soluble polyamino amides prepared for example by polycondensation of an acidic compound with a polyamine; and wherein these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; and wherein these polyamino amides can be alkylated or, if they contain at least one tertiary amine function, they can be quaternized. Such polymers are described, for instance, in French Patent Nos. 2 252 840 and 2 368 508.

(7) The polyamino amide derivatives resulting from the condensation of polyalkylene polyamines with polycarboxylic acids followed by alkylation with difunctional agents. Non-limiting mention may be made, for example, of adipic acid/dialkylaminohydroxyalkyldialkylenetriamine polymers wherein the alkyl radical comprises from 1 to 4 carbon atoms, for example methyl, ethyl and propyl. Such polymers are described for instance in French Patent No. 1 583 363.

Among these derivatives, non-limiting mention may be made of the adipic acid/dimethylaminohydroxypropyl/diethylenetriamine polymers sold under the name CARTARETINE F, F4 or F8 by the company Sandoz.

(8) The polymers obtained by reaction of a polyalkylene polyamine containing two primary amine groups and at least one secondary amine group with a dicarboxylic acid chosen from diglycolic acid and saturated aliphatic dicarboxylic acids comprising 3 to 8 carbon atoms. The mole ratio between the polyalkylene polyamine and the dicarboxylic acid ranges from 0.8:1 to 1.4:1; the polyamino amide resulting therefrom is reacted with epichlorohydrin in a mole ratio of epichlorohydrin relative to the secondary amine group of the polyamino amide ranging from 0.5:1 to 1.8:1. Such polymers are described for instance in U.S. Pat. Nos. 3,227,615 and 2,961,347.

Polymers of this type are sold for example under the name HERCOSETT 57 by the company Hercules Inc. or alternatively under the name PD 170 or DELSETTE 101 by the company Hercules in the case of the adipic acid/epoxypropyl/diethylenetriamine copolymer.

(9) Cyclopolymers of alkyldiallylamine or of dialkyldiallylammonium, such as the homopolymers or copolymers containing, as main constituent of the chain, units chosen from those of formulae (VIII) and (IX):

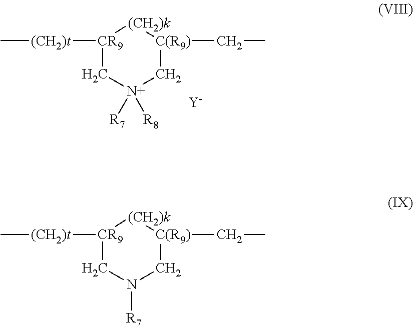

wherein k and t are equal to 0 or 1, and wherein the sum k+t is 1; each instance of R₉ is independently chosen from a hydrogen atom and a methyl radical; R₇ and R₈, are each independently chosen from C₁-C₆ alkyl groups, hydroxyalkyl groups wherein the alkyl group contains for example 1 to 5 carbon atoms, and lower (C₁-C₄) amidoalkyl groups, or R₇ and R₈ may form, together with the nitrogen atom to which they are attached, heterocyclic groups, such as piperidyl or morpholinyl; and according to at least one embodiment, R₇ and R₈, are each independently chosen from C₁-C₄ alkyl groups; Y⁻ is an anion chosen from bromide, chloride, acetate, borate, citrate, tartrate, bisulfate, bisulfite, sulfate and phosphate. These polymers are disclosed for example in French Patent No. 2 080 759 and in its Certificate of Addition No. 2 190 406.

Among the polymers defined above, non-limiting mention may be made for example of the dimethyldiallylammonium chloride homopolymer sold under the name MERQUAT 100 by the company Nalco (and its homologues of low weight-average molecular mass) and the copolymers of diallyldimethylammonium chloride and of acrylamide, sold under the names MERQUAT 550 and MERQUAT 7SPR.

(10) The quaternary diammonium polymer containing repeating units chosen from those of formula (X):

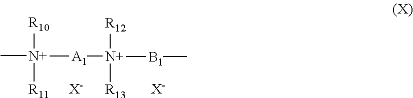

wherein:

R₁₀, R₁₁, R₁₂ and R₁₃, are each independently chosen from aliphatic, alicyclic and arylaliphatic radicals comprising 1 to 6 carbon atoms, and lower hydroxyalkylaliphatic radicals, or alternatively R₁₀, R₁₁, R₁₂ and R₁₃, together or separately, form, with the nitrogen atoms to which they are attached, heterocycles optionally comprising a second heteroatom other than nitrogen, or alternatively R₁₀, R₁₁, R₁₂ and R₁₃ may be chosen from linear and branched C₁-C₆ alkyl radicals substituted with at least one group chosen from nitrile, ester, acyl, amide, and a group —CO—O—R₁₄-D or —CO—NH—R₁₄-D wherein R₁₄ is an alkylene and D is a quaternary ammonium group;

A₁ and B₁ are each independently chosen from linear and branched, saturated and unsaturated polymethylene groups comprising 2 to 8 carbon atoms, and which may contain, linked to or intercalated in the main chain, at least one aromatic ring or at least one oxygen or sulfur atom, or at least one group chosen from sulfoxide, sulfone, disulfide, amino, alkylamino, hydroxyl, quaternary ammonium, ureido, amide and ester groups, and

X⁻ is an anion derived from a mineral or organic acid;

In the alternative, A₁, R₁₀ and R₁₂ can form, with the two nitrogen atoms to which they are attached, a piperazine ring; in addition, if A₁ is chosen from linear and branched, saturated and unsaturated alkylene and hydroxyalkylene radicals, B₁ can also be chosen from —(CH₂)ₙ—CO-D-OC—(CH₂)ₙ— wherein D is chosen from:

a) glycol residues of formula: —O—Z—O—, wherein Z is chosen from linear and branched hydrocarbon-based radicals and groups chosen from one of the following formulae:

—(CH₂—CH₂—O)ₓ—CH₂—CH₂—, and

—[CH₂—CH(CH₃)—O]_{y}—CH₂—CH(CH₃)—,

wherein x and y are integers ranging from 1 to 4, and represent a defined and unique degree of polymerization, or x and y are any number ranging from 1 to 4, and represent an average degree of polymerization;

b) bis-secondary diamine residues such as a piperazine derivative;

c) bis-primary diamine residues of formula: —NH—Y—NH—, wherein Y is chosen from linear and branched hydrocarbon-based radicals, and divalent radicals of formula

—CH₂—CH₂—S—S—CH₂—CH₂—; and

d) ureylene groups of formula: —NH—CO—NH—.

In at least one embodiment of the present disclosure, X¹ is an anion such as chloride or bromide.

These polymers have, for example, a number-average molecular mass ranging from 1,000 to 100,000.

Suitable polymers of this type include, but are not limited to those disclosed in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Other useful polymers include those that are formed from repeating units chosen from those of formula (XI):

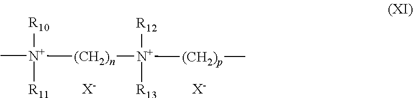

wherein R₁₀, R₁₁, R₁₂ and R₁₃, are each independently chosen from alkyl and hydroxyalkyl radicals comprising 1 to 4 carbon atoms, n and p are integers ranging from 2 to 8, and X¹ is an anion derived from a mineral or organic acid. Non-limiting mention may be made, for example of MEXOMER PO sold by the company Chimex.

(11) Polyquaternary ammonium polymers formed from repeating units of formula (XII):

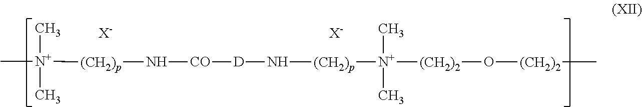

wherein p is an integer ranging from 1 to 6, D is chosen from a direct bond and —(CH₂)ᵣ—CO— wherein r is a number equal to 4 or 7, and X⁻ is an anion.

Such polymers may be prepared according to the processes disclosed in U.S. Pat. Nos. 4,157,388, 4,702,906 and 4,719,282, and in European Patent Application No. 0 122 324.

Among these products, non-limiting examples include MIRAPOL A 15, MIRAPOL AD1, MIRAPOL AZ1 and MIRAPOL 175 sold by the company Miranol.

(12) Quaternary polymers of vinylpyrrolidone and of vinylimidazole, for instance the products sold under the names LUVIQUAT FC 905, FC 550 and FC 370 by the company BASF. These polymers may also comprise other monomers, for instance diallyldialkylammonium halides. Non-limiting mention may be made for instance of the product sold under the name LUVIQUAT SENSATION by the company BASF.

(13) Polyamines such as POLYQUART H sold by Henkel, which is given under the reference name Polyethylene glycol (15) Tallow Polyamine in the CTFA dictionary, or oxyethylenated (15 OE) coconut polyamines.

Other suitable examples of the at least one cationic polymer that may be used according to the present disclosure include, but are not limited to polyalkyleneimines, such as polyethyleneimines, polymers containing vinylpyridine or vinylpyridinium units, condensates of polyamines and of epichlorohydrin, quaternary polyureylenes and chitin derivatives.

In at least one embodiment of the present disclosure, the at least one cationic polymer is chosen from the polymers of families (1), (2), (3), (4), (9), (10) and (12) described above.

In at least one embodiment of the present disclosure, the at least one cationic polymer is chosen from cationic celluloses, cationic guar gums and quaternary polymers of vinylpyrrolidone and of vinylimidazole optionally combined with other monomers.

In at least one embodiment of the present disclosure, the at least one cationic polymer is chosen from hydroxyalkylcelluloses, such as hydroxymethyl-, hydroxyethyl- and hydroxypropyl-celluloses grafted for instance with a methacryloylethyltrimethylammonium, methylacrylamidopropyltrimethylammonium or dimethyldiallylammonium salt, cationic guar gums, and also the copolymer of vinylpyrrolidone and vinylimidazole and dimethyldiallylammonium chloride.

In at least one embodiment, the at least one cationic polymer is chosen from cationic celluloses.

The at least one cationic polymer is present in the compositions according to the present disclosure in a total amount ranging from 0.01% to 5% by weight relative to the total weight of the composition, such as ranging from 0.1% to 1% by weight, for example ranging from 0.15% to 0.5% by weight relative to the total weight of the composition.

In at least one embodiment, the weight ratio between the at least one organosilicon compound and the at least one cationic polymer has a value that ranges from 0.1 to 20, such as ranges from 0.5 to 10, in the cosmetic compositions according to the present disclosure.

According to another embodiment, the weight ratio between the at least one organosilicon compound of formula (III) and the at least one cationic cellulose, such as the cationic hydroxyalkylcelluloses, has a value that ranges from 0.1 to 20.

The composition according to the present disclosure may further comprise at least one additional surfactant chosen from amphoteric surfactants and nonionic surfactants.

Suitable examples of amphoteric or zwitterionic surfactants include, but are not limited to, secondary and tertiary aliphatic amine derivatives wherein the aliphatic group is chosen from linear and branched chains comprising 8 to 22 carbon atoms and comprising at least one anionic group, for instance a carboxylate, sulfonate, sulfate, phosphate or phosphonate group. Non-limiting mention may be made for example of (C₈-C₂₀)alkylbetaines, sulfobetaines, (C₈-C₂₀)alkylamido(C₆-C₈)alkylbetaines or (C₈-C₂₀)alkylamido(C₆-C₈)alkylsulfobetaines.

Among the amine derivatives, mention may be made, in a non-limiting manner, of the products sold under the name MIRANOL®, such as disclosed in U.S. Pat. No. 2,528,378 and U.S. Pat. No. 2,781,354 and classified in the CTFA dictionary, 3rd edition, 1982, under the names Amphocarboxyglycinate and Amphocarboxypropionate, having the respective structures (XIII) and (XIV):

Rₐ—CONHCH₂CH₂—N(R_{b})(R_{c})(CH₂COO⁻) (XIII)

wherein:

Rₐ is chosen from alkyl groups derived from an acid Rₐ—COOH present in hydrolysed coconut oil, heptyl, nonyl and undecyl group,

R_{b} is β-hydroxyethyl group, and

R_{c} is a carboxymethyl group;

and

Rₐ′—CONHCH₂CH₂—N(B)(B′) (XIV)

wherein:

B is —CH₂CH₂OX′,

B′ is —(CH₂)_{z}—Y′, wherein z=1 or 2,

X′ is chosen from —CH₂CH₂—COOH and a hydrogen atom,

Y′ is chosen from —COOH and —CH₂—CHOH—SO₃H,

Rₐ′ is chosen from alkyl groups derived from an acid Rₐ′—COOH present in coconut oil or in hydrolysed linseed oil, alkyl groups, such as a C₁₇ alkyl group, and its iso form, and an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylamphodipropionate, disodium caprylamphodipropionate, lauroamphodipropionic acid, cocoamphodipropionic acid.

By way of example, non-limiting mention may be made of the cocoamphodiacetate sold by the company Rhodia under the trade name MIRANOL® C2M Concentrate.

According to at least one embodiment of the present disclosure, the amphoteric or zwitterionic surfactants can be chosen from (C₈₋₂₀ alkyl)betaines and (C₈₋₂₀ alkyl)amido(C₆₋₈ alkyl)betaines, and mixtures thereof.

When present, the amount of the at least one amphoteric or zwitterionic surfactant is present in an amount ranging from 0.1% to 15% by weight, such as ranging from 0.5% to 10% by weight, for instance, ranging from 1% to 8% by weight relative to the total weight of the composition.

Suitable examples of nonionic surfactants that may be used in the compositions of the present disclosure include those described, for example, in the Handbook of Surfactants by M. R. Porter, published by Blackie & Son (Glasgow and London), 1991, pp. 116-178. They are chosen for instance from polyethoxylated, polypropoxylated and polyglycerolated fatty acids, (C₁-C₂₀)alkylphenols, α-diols and alcohols, having a fatty chain comprising, for example, 8 to 18 carbon atoms, wherein the number of ethylene oxide or propylene oxide groups ranges from 2 to 50, and the number of glycerol groups ranges from 2 to 30.

Non-limiting mention may also be made of condensates of ethylene oxide and of propylene oxide with fatty alcohols; polyethoxylated fatty amides having from 2 to 30 ethylene oxide units, polyglycerolated fatty amides containing on average 1 to 5, for instance 1.5 to 4 ethylene oxide units, ethoxy related fatty acid esters of sorbitan containing from 2 to 30 ethylene oxide units, fatty acid esters of sucrose, fatty acid esters of polyethylene glycol, (C₆₋₂₄ alkyl)polyglycosides, N—(C₆₋₂₄ alkyl)glucamine derivatives, amine oxides such as (C₁₀-C₁₄)alkylamine oxides or N—(C₁₀₋₁₄ acyl)aminopropylmorpholine oxides.

When present, the amount of the at least one additional nonionic surfactant is present in an amount ranging from 0.01% to 10% by weight, such as ranging from 0.1% to 10% by weight relative to the total weight of the composition.

According to at least one embodiment, the cosmetic composition further comprise at least one amphoteric surfactants as an additional surfactant.

In at least one embodiment of the present disclosure, the total amount of surfactants ranges from 3% to 50% by weight, such as ranges from 5% to 30% by weight, for instance ranges from 8% to 20% by weight relative to the total weight of the cosmetic composition.

The cosmetic compositions according to the present disclosure may further comprise at least one organic acid.

As used herein, “organic acid” is understood to mean any non-polymeric organic compound comprising at least one acid functional group chosen from carboxylic acid, sulfonic acid and phosphoric acid functional groups.

In at least one embodiment, the at least one organic acid is not a surfactant.

According to another embodiment, the molecular weight of the at least one organic acid is less than 250, such as less than 200.

In at least one embodiment of the present disclosure, the at least one organic acid is chosen from amino acids.

The at least one organic acid may also be chosen from acetic acid, propanoic acid, butanoic acid, lactic acid, malic acid, glycolic acid, ascorbic acid, maleic acid, phthalic acid, succinic acid, taurine, tartaric acid, arginine, glycine, glucuronic acid, gluconic acid and citric acid.

According to at least one embodiment, the at least one organic acid is chosen from carboxylic acids, such as α-hydroxylated carboxylic acids or AHAs.

In at least one embodiment, the at least one organic acid used in the compositions disclosed herein is chosen from lactic acid and citric acid, such as, lactic acid.

In the compositions disclosed herein, the at least one organic acid may be in free or salified form.

The at least one organic acid that may be used in the compositions according to the present disclosure may be present in an amount, expressed as free acids, ranging from 0.01% to 10% by weight, such as ranging from 0.1% to 8% by weight, for example ranging from 0.2% to 5% by weight relative to the total weight of the composition.

The cosmetic compositions according to the present disclosure may further comprise at least one silicone, such as amino silicones.

As used herein, “amino silicone” is understood to mean any silicone comprising at least one primary, secondary or tertiary amine function or a quaternary ammonium group.

Suitable amino silicones include, but are not limited to:

(a) compounds chosen from those of formula (XV):

(R¹)ₐ(T)₃₋ₐ-Si[OSi(T)₂]ₙ-[OSi(T)_{b}(R¹)_{2-b}]ₘ—OSi(T)₃₋ₐ-(R¹)ₐ (XV)

wherein:

T is chosen from a hydrogen atom, phenyl, hydroxyl (—OH), and C₁-C₈ alkyl radicals, and in at least one embodiment, T is chosen from methyl and C₁-C₈ alkoxy groups, for instance methoxy,

a is 0 or an integer ranging from 1 to 3, and in at least one embodiment, 0,

b is 0 or 1, and in at least one embodiment, 1,

m and n are numbers such that the sum (n+m) can range from 1 to 2,000, such as range from 50 to 150, and wherein n is a number ranging from 0 to 1999, for instance, ranging from 49 to 149, and wherein m is a number ranging from 1 to 2000, for example, ranging from 1 to 10;

R¹ is chosen from monovalent radicals of formula —C_{q}H_{2q}L wherein q is a number ranging from 2 to 8 and L is chosen from optionally quaternized amino groups chosen from the following groups:

—N(R²)—CH₂—CH₂—N(R²)₂;

—N(R²)₂;

—N⁺(R²)₃Q⁻;

—N⁺R²)(H)₂Q⁻;

—N⁺R²)₂HQ⁻; and

—N(R²)—CH₂—CH₂—N⁺(R²)(H)₂Q⁻,

wherein R² is chosen from a hydrogen atom, phenyl, benzyl, and saturated monovalent hydrocarbon-based radicals, for example C₁-C₂₀ alkyl radicals, and Q⁻ is a halide ion such as fluoride, chloride, bromide or iodide.

In at least one embodiment of the present disclosure, the at least one amino silicone corresponding to the definition of formula (XV) is chosen from compounds of formula (XVI):

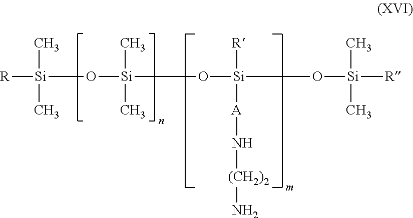

wherein R, R′ and R″, are each independently chosen from C₁-C₄ alkyl radicals, for example CH₃; C₁-C₄ alkoxy radicals, such as methoxy; and OH; A is chosen from linear and branched, C₃-C₈ alkylene radicals, such as C₃-C₆ alkylene radicals; m and n are integers dependent on the molecular weight and whose sum ranges from 1 to 2,000.

According to at least one embodiment of the present disclosure, R, R′ and R″, are each independently chosen from C₁-C₄ alkyls and hydroxyl radicals, A is a C₃ alkylene radical and m and n are such that the weight-average molecular mass of the compound ranges from 5,000 to 500,000. Compounds of this type are referred to in the CTFA dictionary as Amodimethicones.

According to at least one embodiment, R, R′ and R″, are each independently chosen from C₁-C₄ alkoxy and hydroxyl radicals, and at least one of the radicals R or R″ is an alkoxy radical, and A is a C₃ alkylene radical. The hydroxyl/alkoxy mole ratio ranges, for example, from 0.2/1 to 0.4/1, and in at least one embodiment, is equal to 0.3/1. Moreover, m and n are such that the weight-average molecular mass of the compound ranges from 2000 to 10⁶. In at least one embodiment, n ranges from 0 to 999 and m ranges from 1 to 1000, wherein the sum of n and m ranges from 1 to 1000.

In this category of compounds, non-limiting mention may be made, for example, of the product BELSIL® ADM 652 sold by Wacker.

According to at least one embodiment, R and R″, are each independently chosen from C₁-C₄ alkoxy and hydroxyl radicals, and at least one of the radicals R or R″ is an alkoxy radical, R′ is a methyl radical and A is a C₃ alkylene radical. The hydroxyl/alkoxy mole ratio ranges for instance from 1/0.8 to 1/1.1, and in at least one embodiment, is equal to 1/0.95. Moreover, m and n are such that the weight-average molecular mass of the compound ranges from 2,000 to 200,000. According to at least one embodiment, n ranges from 0 to 999 and m ranges from 1 to 1000, wherein the sum of n and m ranges from 1 to 1000.

In at least one embodiment, non-limiting mention may be made of the product FLUID WR® 1300 sold by Wacker.

It should be noted that the molecular mass of these silicones is determined by gel permeation chromatography (ambient temperature, polystyrene standard; μ styragem columns; eluent THF; flow rate 1 mm/m; 200 μl of a solution containing 0.5% by weight of silicone are injected into THF and detection is performed by UV refractometry).

A product corresponding to the definition of formula (XV) is for example the polymer known in the CTFA dictionary as Trimethylsilyl Amodimethicone, corresponding to formula (XVIII) below:

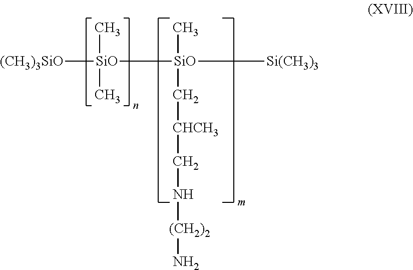

wherein n and m have the meanings given above in accordance with formula (XV).

Such compounds are described, for example, in European Patent Application No. 0 95 238; a compound of formula (XVIII) is sold, for example, under the name Q2-8220 by the company OSI.

(b) compounds chosen from those of formula (XIX) below:

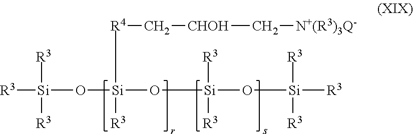

wherein:

each instance of R³ is chosen from monovalent C₁-C₁₈ hydrocarbon-based radicals, for instance, C₁-C₁₈ alkyl and C₂-C₁₈ alkenyl radicals, for example methyl;

R⁴ is chosen from divalent hydrocarbon-based radicals, such as C₁-C₁₈ alkylene radicals and divalent C₁-C₁₈ radicals, and for example C₁-C₈ alkyleneoxy radicals;

Q⁻ a is a halide ion, for instance chloride;

r is an average statistical value ranging from 2 to 20, such as ranging from 2 to 8;

s is an average statistical value ranging from 20 to 200, for example ranging from 20 to 50.

Such compounds are described for instance in U.S. Pat. No. 4,185,087.

A non-limiting example of a compound falling within this class is the product sold by the company Union Carbide under the name UCAR SILICONE ALE 56.

(c) the quaternary ammonium silicones of formula (XX):

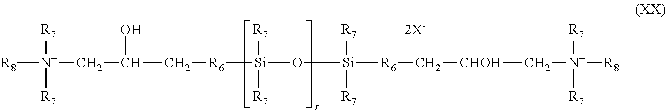

wherein:

each instance of R₇, is independently chosen from monovalent hydrocarbon-based radicals comprising 1 to 18 carbon atoms, for example C₁₀-C₁₈ alkyl radicals; C₂-C₁₈ alkenyl radicals; and rings comprising 5 or 6 carbon atoms, for example methyl;

each instance of R₆, is independently chosen from divalent hydrocarbon-based radicals, such as C₁-C₁₈ alkylene radicals and divalent C₁₀-C₁₈ radicals, for example C₁-C₈, alkyleneoxy radicals linked to the Si via an SiC bond;

each instance of R₈, is independently chosen from a hydrogen atom, and monovalent hydrocarbon-based radicals comprising 1 to 18 carbon atoms, for instance C₁-C₁₈ alkyl radicals, C₂-C₁₈ alkenyl radicals and —R₆—NHCOR₇;

X⁻ is an anion such as a halide ion, for example chloride, or an organic acid salt, such as acetate;

r is a mean statistical value ranging from 2 to 200, for instance ranging from 5 to 100.

These silicones are described, for example, in European Patent Application No. 0 530 974.

(d) the amino silicones of formula (XXI):

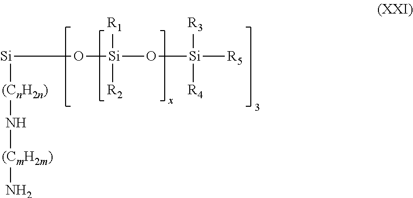

wherein:

R₁, R₂, R₃ and R₄, are each independently chosen from C₁-C₄ alkyl radicals and a phenyl group,

R₅ is chosen from C₁-C₄ alkyl radicals and a hydroxyl group,

n is an integer ranging from 1 to 5,

m is an integer ranging from 1 to 5, and

wherein x is chosen such that the amine number ranges from 0.01 to 1 meq./g.

According to at least one embodiment of the present disclosure, the at least one amino silicone does not comprise any quaternary ammonium groups.

As used herein, “cosmetically acceptable medium” is understood to mean a medium that is compatible with keratin fibers, such as the hair.

The cosmetically acceptable medium is formed from water or from a mixture of water and at least one cosmetically acceptable solvent chosen from C₁-C₄ lower alcohols, such as ethanol, isopropanol, tert-butanol and n-butanol; polyols such as glycerol, propylene glycol and polyethylene glycols; and mixtures thereof.

The pH of the compositions according to the present disclosure ranges from 3 to 11, for instance ranges from 5 to 9.

The composition according to the present disclosure may further comprise at least one additive known in the art, such as natural and synthetic thickeners; viscosity regulators; C₁₂-C₃₀ fatty alcohols; ceramides; oily fatty esters such as isopropyl myristate, myristyl myristate, cetyl palmitate and stearyl stearate; mineral, plant and synthetic oils such as α-olefins or avocado oil, rapeseed oil, apricot oil, camellina oil and liquid petroleum jelly; vitamins and provitamins; amphoteric polymers; pH stabilizers, preserving agents; and dyes.

The at least one thickener may be chosen from cellulose-based thickeners, for example hydroxyethylcellulose, hydroxypropylcellulose and carboxymethylcellulose, guar gum and derivatives thereof, for example the hydroxypropyl guar sold by the company Rhodia under the reference JAGUAR HP 105, gums of microbial origin, such as xanthan gum and scleroglucan gum, synthetic thickeners such as crosslinked acrylic acid or acrylamidopropanesulfonic acid homopolymers, for example CARBOMER, nonionic, anionic, cationic or amphoteric associative polymers, such as the polymers sold under the names PEMULEN TR1 or TR2 by the company Goodrich, SALCARE SC90 by the company Ciba, ACULYN 22, 28, 33, 44 or 46 by the company Rohm & Haas, and ELFACOS T210 and T212 by the company Akzo.

A person skilled in the art will take care to select the optional additives and the amount thereof such that they do not harm the properties of the compositions of the present disclosure.

The at least one optional additive may be present in the composition according to the present disclosure in an amount ranging from 0 to 20% by weight relative to the total weight of the composition.

In at least one embodiment, the cosmetic compositions disclosed herein are transparent or translucent, for example allowing a transmittance at 600 nm of greater than 85%, such as greater than 90%, for example greater than 94%.

The compositions in accordance with the present disclosure may be in the form of shampoos for washing and conditioning the hair, and are, for example applied to wet hair in amounts that are effective for washing them, and the lather generated by massaging or frictioning with the hands may then be removed, after an optional leave-on time, by rinsing with water, the operation possibly being repeated at least one time.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The following examples serve to illustrate embodiments of the present disclosure without, however, exhibiting a limiting nature.

### EXAMPLE

Composition (A) in accordance with the present disclosure was prepared as set forth in the table below. The amounts are expressed as weight percentages of product in the given form, relative to the total weight of the composition.

| | A |
|---|---|
| Composition | (invention) |
| Lactic acid | 0.27 |
| Mixture of chloro-5-methyl-2-isothiazolin-4-one- | 0.1 |
| 3/methylisothiazolin-4-one-3/magnesium chloride | |
| and nitrate in aqueous solution ⁽¹⁾ | |
| Polyethoxylated (55 EO) propylene glycol oleate and | 0.6 |
| propylene glycol oleate as a water-glycol solution ⁽²⁾ | |
| Hydroxyethylcellulose quatemized with | 0.6 |
| 2,3-epoxypropyltrimethylammonium chloride | |
| at 95% AM ⁽³⁾ | |
| Polydimethylsiloxane containing aminoethyl | 1 |
| aminoisobutyl and trimethylsiloxy groups ⁽⁴⁾ | |
| 3-Aminopropyltriethoxysilane ⁽⁵⁾ | 0.75 |
| Cocoylbetaine at 30% AM in aqueous solution ⁽⁶⁾ | 17 |
| Oxyethylenated (20 OE) and oxypropylenated (5 | 0.5 |
| OP) cetyl alcohol ⁽⁷⁾ | |
| Lauryl ether carboxylic acid (4.5 OE) at 90% active | 1 |
| material in water ⁽⁸⁾ | |
| Coconut acid monoisopropanolamide at 94.5% | 0.85 |
| active material ⁽⁹⁾ | |
| Sodium lauryl ether sulfate (2.2 OE) as an aqueous | 16 |
| solution (70% AM) ⁽¹⁰⁾ | |
| pH regulator | qs pH = 9 |
| Fragrance | 0.5 |
| Deionized water | qs 100 g |

⁽¹⁾ sold under the trade name KATHON CG by the company Rohm & Haas
⁽²⁾ sold under the trade name ANTIL 141 Liquid by the company Evonik Goldschmidt
⁽³⁾ sold under the trade name POLYQUAT 400 KC by the company KCI
⁽⁴⁾ sold under the trade name DC 28566 Amino Fluid by the company Dow Corning
⁽⁵⁾ sold under the name XIAMETER OFS EO11 Silane by the company Dow Corning
⁽⁶⁾ sold under the name MIRATAINER BB/FLA by the company Rhodia
⁽⁷⁾ sold under the name PROCETYL AWS-LQ by the company Croda
⁽⁸⁾ sold under the name AKYPO RLM 45 CA by the company Kao
⁽⁹⁾ sold under the name EMPILAN CIS by the company Huntsman
⁽¹⁰⁾ sold under the name TEXAPON AOS 225UP by the company Cognis

A composition that was clear and stable over time was obtained.

When applied as a shampoo, composition (A) afforded satisfactory styling effects, for example, gave the hair body, volume and a satisfactory soft feel.

What is claimed is:

1. A cosmetic composition for washing and conditioning keratin fibers, comprising, in a cosmetically acceptable medium:
(i) at least one organosilicon compound chosen from silanes comprising one silicon atom and siloxanes comprising two or three silicon atoms, and wherein the at least one organosilicon compound also comprises at least one basic chemical functional groups and at least one group chosen from hydroxyl and hydrolysable groups per molecule;
(ii) at least one anionic surfactant; and
(iii) at least one cationic polymer.

2. The cosmetic composition according to claim 1, wherein the at least one basic chemical functional group of the organosilicon compound is chosen from primary, secondary and tertiary amines.

3. The cosmetic composition according to claim 1, wherein the at least one hydrolysable group is chosen from alkoxy, aryloxy and halogen groups.

4. The cosmetic composition according to claim 1, wherein the at least one organosilicon compound is chosen from compounds of formula (I): 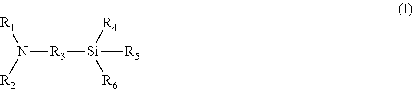
wherein:
R₄ is chosen from halogen atoms, OR′, and R′₁;
R₅ is chosen from halogen atoms, OR″, and R′₂;
R₆ is chosen from halogen atoms, OR′″, and R′₃;
R₁, R₂, R₃, R′, R″, R′″, R′₁, R′₂ and R′₃ are each independently chosen from saturated and unsaturated, linear and branched hydrocarbon-based groups optionally bearing additional chemical groups; or in the alternative, R₁, R₂, R′, R″ and R′″ may also be a hydrogen atom, and wherein at least two of the groups R₄, R₅ and R₆ are chosen from OR′, OR″ and OR′″, and at least two of the groups R′, R″ and R′″ are other than hydrogen; and 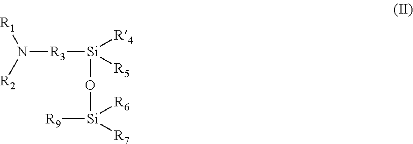
wherein:
R₁, R₂, R₃, R₅ and R₆ are as defined previously;
R′₄ is chosen from halogen atoms, and OR₁₁;
R₇ is chosen from halogen atoms, OR₁₀, and R″₁;
R₉ is chosen from halogen atoms, OR₈, R″₂ and R₃NR₁R₂; and
R″₁, R″₂, R₈, R₁₀ and R₁₁ are each independently chosen from saturated and unsaturated, linear and branched hydrocarbon-based groups, optionally bearing additional chemical groups, or in the alternative, R₁₁, R₁₀ and R₈ may also be a hydrogen atom; and wherein at least one of the groups R₆, R₇ and R₉ is chosen from halogen atoms, OR′″, OR₁₀ and OR₈.

5. The cosmetic composition according to claim 4, wherein R₁, R₂, R′, R′₁, R′₂, R′₃, R″, R′″, R″₁, R″₂, R₈, R₁₀ and R₁₁ are chosen from C₁-C₁₂ alkyl, C₆-C₁₄ aryl, (C₁-C₈)alkyl(C₆-C₁₄)aryl and (C₆-C₁₄)aryl(C₁-C₈)alkyl radicals.

6. The cosmetic composition according to claim 1, wherein the at least one organosilicon compound is chosen from compounds of formula (III): 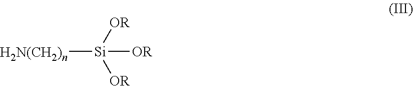
wherein each instance of R is independently chosen from C₁-C₆ alkyl radicals, and n is an integer ranging from 1 to 6.

7. The cosmetic composition according to claim 1, wherein the at least one anionic surfactant is chosen from alkyl sulfates, alkyl ether sulfates and alkyl ether carboxylates, and mixtures thereof.

8. The cosmetic composition according to claim 1, wherein the at least one cationic polymer is chosen from cationic celluloses, cationic guar gums and quaternary polymers of vinylpyrrolidone and of vinylimidazole optionally combined with other monomers.

9. The cosmetic composition according to claim 1, wherein the at least one cationic polymer is chosen from cationic celluloses.

10. The cosmetic composition according to claim 1, wherein the weight ratio of the at least one organosilicon compound (i) and the at least one cationic polymer (iii) has a value ranging from 0.1 to 20.

11. The cosmetic composition according to claim 1, further comprising at least one component chosen from amphoteric surfactants and silicones.

12. A cosmetic process for treating keratin fibers, comprising applying to keratin fibers a cosmetic composition comprising in a cosmetically acceptable medium:
(i) at least one organosilicon compound chosen from silanes comprising one silicon atom and siloxanes comprising two or three silicon atoms, and wherein the at least one organosilicon compound also comprises at least one basic chemical functional groups and at least one group chosen from hydroxyl and hydrolysable groups per molecule;
(ii) at least one anionic surfactant; and
(iii) at least one cationic polymer; and
wherein the cosmetic composition is rinsed out after an optional leave-on time.

13. The cosmetic composition according to claim 1, wherein the cosmetic composition is in the form of a shampoo for cleansing and conditioning keratin fibers.

14. The cosmetic composition according to claim 6, wherein n is an integer ranging from 2 to 4.

15. The cosmetic composition according to claim 7, wherein the at least one anionic surfactant is chosen from alkali metal and alkaline-earth metal, ammonium, amine and amino alcohol salts.

16. The cosmetic composition according to claim 10, wherein the weight ratio of the at least one organosilicon compound (i) and the at least one cationic polymer (iii) has a value ranging from 0.5 to 10.

17. The cosmetic composition according to claim 16, wherein the weight ratio of the at least one organosilicon compound (i) and the at least one cationic polymer (iii) has a value ranging from 0.8 to 5.

18. The cosmetic composition according to claim 11, wherein the at least one component is chosen from amino silicones.
